(19) United States
(12) Patent Application Publication
(10) Pub. No.: US 2022/0202139 A1
(43) Pub. Date: Jun. 30, 2022

(54) CLOSING DEVICE FOR ORTHOSIS

(71) Applicant: ORTHOSERVICE AG, CHIASSO (CH)

(72) Inventors: FRANCESCO TURCONI, MONZA (IT); PAOLO ROSSI, MORBIO INFERIORE (CH)

(21) Appl. No.: 17/557,319

(22) Filed: Dec. 21, 2021

(30) Foreign Application Priority Data
Dec. 28, 2020 (IT) 102020000032502

(51) Int. Cl. A44B 11/04; A44B 18/00; A61F 5/01

(52) U.S. Cl. CPC A44B 11/04; A61F 5/0109; A44B 18/00

(57) ABSTRACT

A closing device for orthosis including a buckle and a strap engaged to each other, respectively associable at their opposite ends to portions of orthosis. The buckle includes at least two rings between which a wall is placed, which is provided with a hook-shaped quick coupling element on the upper surface. The strap includes on one side a portion of a loop-shaped quick coupling element for gripping on the relative quick coupling element of the wall.

### CROSS-REFERENCED TO RELATED APPLICATIONS

This application is related to and claims the benefit of Italian Patent Application No. 102020000032502, filed on Dec. 28, 2020, the contents of which are herein incorporated by reference in their entirety.

### TECHNICAL FIELD

The present disclosure relates to a closing device for an orthosis which requires initially adjusting the length of a strap and then closing the orthosis under tension to the predetermined length, as is the case for example in a hyperextension brace or a knee brace.

### BACKGROUND

Especially in the hyperextension brace, the adjustment of the rear belt is particularly critical. The belt is typically made of an inextensible fabric strap (like a safety belt). For example, as shown in EP 2457544, this is coupled on one side to a lever that enables easily closing the product after the length has been adjusted the first time; on the opposite side, the belt is inserted in a buckle that engages to the metal part by means of a mushroom-shaped hook. As said, the length of the belt must therefore be adjusted the first time and thereafter this adjustment (performed by the technician) must not be changed accidentally.

For this reason, a number of systems have been set up over time to carry out this operation.

The most commonly used system provides a so-called double-pass “loop buckle”, by means of which the belt is “choked” and locked by friction. The problem with this solution is that if the belt is not fully tensioned (as it can occur before closing the lever on the opposite side), the adjustment may be lost. For this reason, a loop-shaped quick coupling means (such as Velcro®) is sewn throughout the length of the belt and a hook-shaped quick coupling means (such as Velcro®) is sewn at the final part, so that the adjustment can be in any case “fastened”. The disadvantage of the latter solution is, in turn, that it has to be closed at the rear part/on the patient's back, which turns out to be difficult when the patient is bedridden (a normal situation in which the product is applied).

An alternative system is the triple-pass loop buckle. By this system the belt is locked in the same way as in the previous system, but with the advantage that there is no need for quick-coupling means (such as Velcro®), as the belt engaged in the triple passage does not slide accidentally even when the belt is loosened. The disadvantage of this solution is that passing the belt through the three slots of the buckle is difficult and time-consuming, as it has to be shortened a little at a time; in addition, the exceeding strap has to be fastened in any case, for example by a fastening clip, so that it is not left free during use.

A further system is the one described in EP2457544. In this case the adjustment is made with the toothed belt system, which may however require the use of a fastening screw to be adjusted by means of a screwdriver in order to fasten it once the length has been established, taking into account the large forces involved.

### SUMMARY

The present disclosure provides a closing device for an orthosis that overcomes the drawbacks of the prior art.

The present disclosure also provides a closing device for an orthosis, which is particularly simple and functional, with reduced costs.

These advantages according to the present disclosure are reached by providing a closing device for an orthosis as recited in claim 1.

Further features are comprised in the dependent claims.

### BRIEF DESCRIPTION OF THE DRAWINGS

The features and advantages of the closing device for an orthosis according to the present disclosure will be more apparent from the following exemplifying and not limiting description, referred to the schematic attached drawings, wherein:

FIG. 1 shows a closing device for an orthosis according to the disclosure during the first closing steps;

FIG. 2 shows a detail of the device of FIG. 1;

FIG. 3 shows the device of FIG. 1 during the subsequent closing steps; and

FIG. 4 shows the device of FIG. 1 in a closed position.

### DETAILED DESCRIPTION OF THE DRAWINGS

With reference to the figures, a closing device 10 for an orthosis comprising a loop buckle 20 and a strap 30, placed in mutual engagement and respectively associable with portions of the orthosis, capable of being maintained in tension in a mutual predetermined position is shown.

The loop buckle 20 comprises an adjustment and fastening end 21 of the strap 30 and an opposite end 22 adapted to be constrained to the orthosis, not shown, for example by means of a further strap 23 and/or loop 24.

The adjustment and fastening end 21 preferably comprises two rings 25, 25′, facing respectively the body of the buckle 20 and the outside of the buckle 20, identifying as many slots, between which a wall 26 is placed, which is provided on the upper surface with hook-shaped quick coupling means (such as Velcro®), which may be glued, riveted or directly moulded on the piece.

A double-pass loop buckle is thereby made; a triple-pass loop buckle comprising three rings would also form part of the disclosure.

Preferably, the outer ring 25′ is provided with an opening 27 for ease of use.

The strap 30, preferably made of inextensible fabric, comprises, on one side, a portion 31 of loop-shaped quick coupling means (such as Velcro®) adapted to be folded on itself, for gripping the wall 26 provided with the relative quick coupling means.

Preferably, at the free end, the strap 30 includes a portion 32 of hook-shaped quick coupling means (such as Velcro®) for firmly holding it in a position folded on itself when closed.

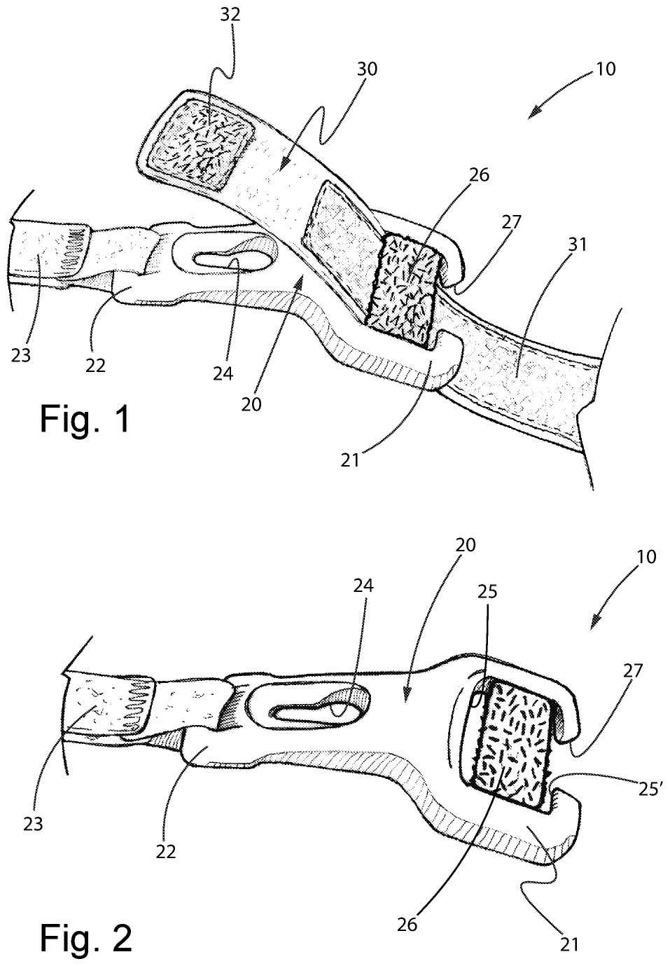

In order to close the closing device for an orthosis, which is an advantage of the present disclosure, the end of the strap 30 is inserted into the first ring 25 of the buckle 20 from the bottom upwards (FIG. 1).

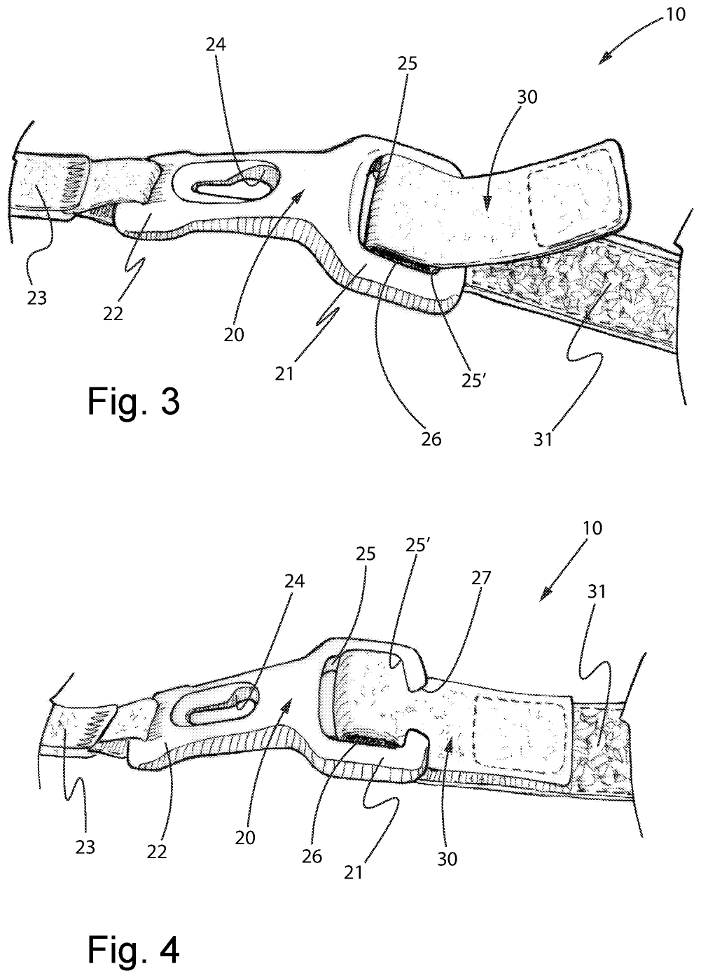

When the strap 30 is placed under tension, the portion 31 of the loop-shaped quick coupling means and the wall 26 provided with hook-shaped quick coupling means are engaged in the subsequent tensioning positions. This allows to tension the strap 30 and immediately pre-fasten it thanks to the engagement between the quick coupling means (FIG. 3).

Once the correct length has been defined, the strap 30 is passed through the second loop 25′, preferably by making use of the opening 27, in order to securely fasten the overall buckle 20 and strap 30 (FIG. 4).

By engaging the portion 32 of the hook-shaped quick coupling means placed at the end of the strap 30 and the portion 31 of loop-shaped quick coupling means, the end of the strap 30 is firmly held in a closed position.

The closing device for an orthosis of the present disclosure has the following advantages:

The strap 30 may be very easily pulled as it is initially passed through only one ring;

Locking is immediate thanks to the use of the quick coupling means;

Then, after inserting it into the second ring, the holding security is obtained and the pre-set length is maintained;

The exceeding part is also fastened by means of quick coupling means.

In particular, when used with a hyperextension brace, this has the advantage over the traditional solution of being able to close the strap not under the back of the bedridden patient, but when the patient stands up, because the system is in any case fastened.

The closing device for an orthosis as conceived herein is susceptible to several modifications and variations, all falling within the disclosure; furthermore, all the details are replaceable by technically equivalent elements. In practice, the materials used, as well as their dimensions, can be of any type according to the technical requirements.

1. A closing device for orthosis comprising: a buckle and a strap engaged to each other, respectively associable at their opposite ends to portions of orthosis, wherein the buckle comprises at least two rings between which a wall is placed, the wall being provided with hook-shaped quick coupling means on the upper surface, and wherein the strap comprises on one side a portion of loop-shaped quick coupling means configured for gripping on the quick coupling means of the wall.

2. The closing device for orthosis according to claim 1, wherein the hook-shaped quick coupling means provided on the wall are glued, riveted, or directly moulded on the piece.

3. The closing device for orthosis according to claim 1, wherein an outer ring of the at least two rings is provided with an opening.

4. The closing device for orthosis according to claim 1, wherein the strap comprises a portion of hook-shaped quick coupling means for firmly holding the strap in a folded position on itself when closed.

5. The closing device for orthosis according to claim 1, wherein the strap is made up of inextensible fabric.
